(19) United States
(12) Patent Application Publication
(10) Pub. No.: US 2009/0298077 A1
(43) Pub. Date: Dec. 3, 2009

(54) ASSAY FOR MEASUREMENT OF APURINIC/APYRIMIDINIC (AP) SITES AND FOR SCREENING AP-SITE REACTIVE COMPOUNDS

(76) Inventors: Yan Xu, Solon, OH (US); Stanton Gerson, Hunting Valley, OH (US); Lili Liu, Macedonia, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/439,626

(22) PCT Filed: Aug. 29, 2007
(86) PCT No.: PCT/US07/77062

§ 371 Date: Jun. 24, 2009

Related U.S. Application Data
(60) Provisional application No. 60/823,804, filed on Aug. 29, 2006.

(51) Int. Cl. C12Q 1/68

(52) U.S. Cl. 435/6

(57) ABSTRACT

A method of detecting abasic (AP) sites in DNA from a subject includes isolating a sample of DNA from a subject under examination, contacting the DNA with a fluorescent aldehyde reactive probe (FARP), and detecting FARP labeled AP sites in the DNA sample.

### RELATED APPLICATIONS

This application claims priority from U.S. Provisional Application No. 60/823,804, filed Aug. 29, 2006, the subject matter, which is incorporated herein by reference.

### FIELD OF THE INVENTION

The present invention relates to an assay for measurement of genomic DNA apurinic/apyrimidinic (AP) sites, and more particularly, to a fluorometric assay for measurement of AP sites of DNA.

### BACKGROUND OF THE INVENTION

Apurinic/apyrimidinic (AP) sites are generated through loss of damaged bases (purine or pyrimidine) in the DNA strands. AP sites exist in equilibrium between the ring-closed and the ring-opened form Unrepaired AP sites are mutagenic and lethal to cells. Therefore, quantitative measurement of AP sites in cellular DNA is crucial for the assessment of DNA damage and repair, as well as for the development of anti-cancer drugs that target the BER pathway (Adamczyk (1998) Bioorganic Med. Chem. Letters 8:3599-3602: Nakamura et al. (1998) Canc. Res. 58:222-225: Sun et al. (2001) Anal. Chem. 73:2229-2232).

The condensation reaction between aldehyde group of ring-opened AP site and nucleophile is the most used reaction for labeling AP site in analytical measurement. In 1992, aldehyde reactive probe (ARP, N′-aminooxymethylcarbonylhydrazino-D-biotin) assay was developed. An ELISA-like assay is commercially available in a 96-well format through Dojindo Molecular Technologies (Gaithersburg, Md., USA), which uses ARP (a biotinylated alkoxyamine) to react specifically with the aldehyde group in the ring-opened AP site. After the reaction of DNA-AP sites with excess amount of ARP reagent, the remaining ARP reagent is washed away with a buffer solution. The resulting ARP-AP complex is incubated with excess amount of horseradish peroxidase-conjugated streptavidin (HRP-streptavidin). The biotin of the ARP-AP complex binds to streptavidin on HRP-streptavidin. After washing away the unbound HRP-streptavidin, the horseradish peroxidase enzyme substrate, 3,3′,5,5′-tetramethylbenzidine (TMB), is added and incubated at 37° C. for 1 hr. The absorbance of each well is read at 650 nm and the number of AP sites in the DNA sample is determined using a calibration curve. Although the assay is very sensitive and uses a small amount of DNA sample (100 ng), the non-specific adsorption of HRP-streptavidin on the surface of the reaction well results in poor assay precision and high background signal.

There have been more recent reports of the determination of AP sites including the matrix-assisted laser desorption ionization mass spectrometry and electrospray ionization tandem mass spectrometry methods for measurement of the hydrazone modified oligodeoxynucleotides, and the atomic force microspray method for detection of ARP modified AP sites (Lindahl and Nyberg (1972) 11:3610-3618), as well as scanning near-field optical microspray (SNOM) for imaging of fluorescence labeled AP sites through the biotin-streptavidin conjugation (Kim et al. (2003) FEBS Letters 555:611-615).

### SUMMARY OF THE INVENTION

The present invention relates to a method of detecting abasic (AP) sites in DNA from a biological sample. The method includes isolating a sample of DNA from the biological sample. The isolated sample is contacted with a fluorescent aldehyde reactive probe (FARP). The FARP labeled AP sites in the DNA sample are then detected.

In an aspect of the invention, the DNA can be extracted from the subject's cells before the contacting the DNA with FARP. The FARP can comprise fluorescein-5-thiosemicarbazide. The number of AP sites in the sample of DNA can be correlated to the number of AP sites in a control DNA specimen. The control DNA specimen can comprise individual AP-DNA standards having known concentrations of AP sites. The number of AP sites of sample DNA and the control DNA specimen can be determined substantially simultaneously. Unbound FARP can be removed from the sample of DNA after contacting the DNA with the FARP.

Another aspect of the invention relates to a method of quantitating AP sites in DNA of a biological sample obtained from a subject. The method includes isolating a sample of DNA. The isolated sample of DNA is contacted with a FARP reagent. Unbound FARP is then removed from the isolated DNA sample. The number of AP sites in the sample of DNA is quantitatively assessed.

In a further aspect, the number of AP sites in the DNA sample can be quantitated by fluorometric analysis. The FARP reagent can include fluorescein-5-thiosemicarbazide. The fluorescence intensity of the sample of DNA can be correlated to the concentration of AP sites in the sample of DNA by comparing the fluorescence intensity in the sample of DNA to the fluorescence intensity of at least one control DNA specimen. The control DNA specimen can comprise an individual AP-DNA standard having a known concentration of AP sites.

The present invention further relates to a kit for assaying a sample of DNA. The kit includes a control DNA specimen having a known concentration of AP sites and a FARP reagent. The control DNA specimen can include an AP-DNA standard having a known concentration of AP-sites.

The present invention also relates to a method of screening therapeutic agents for inhibiting base excision repair (BER). The method includes contacting a sample of AP-DNA with an FARP reagent and one or more therapeutic agents. Unbound FARP reagent and the one or more therapeutic agents is then removed from the sample of AP-DNA. The FARP labeled AP sites in the sample of AP-DNA are subsequently detected. The level or number of FARP labeled AP sites in the sample of AP-DNA are then correlated to or compared with a sample of AP DNA contacted with FARP in the absence of the therapeutic agent. A reduced level of FARP labeled AP sites in the sample of AP-DNA compared to the sample of AP-DNA not containing the therapeutic agent is indicative of an effective therapeutic agent or an effective combination of therapeutic agents.

In an aspect of the invention, the therapeutic agent can comprise a DNA repair inhibitor. The DNA repair inhibitor can include a base excision repair inhibitor. The base excision repair inhibitor can include an AP endonuclease inhibitor. The therapeutic agent can also include a compound capable of forming a covalent linkage with an aldehyde group on AP-DNA.

### BRIEF DESCRIPTION OF THE DRAWINGS

FIG. 1 is a schematic flow diagram of a method in accordance with an aspect of the invention.

FIG. 2 is a schematic flow diagram of a method in accordance with another aspect of the invention.

FIG. 3 illustrates that AP sites in DNA strands exist in equilibrium between ring closed and ring opened form. The ring-open form AP site has an active aldehyde group that can be labeled chemically.

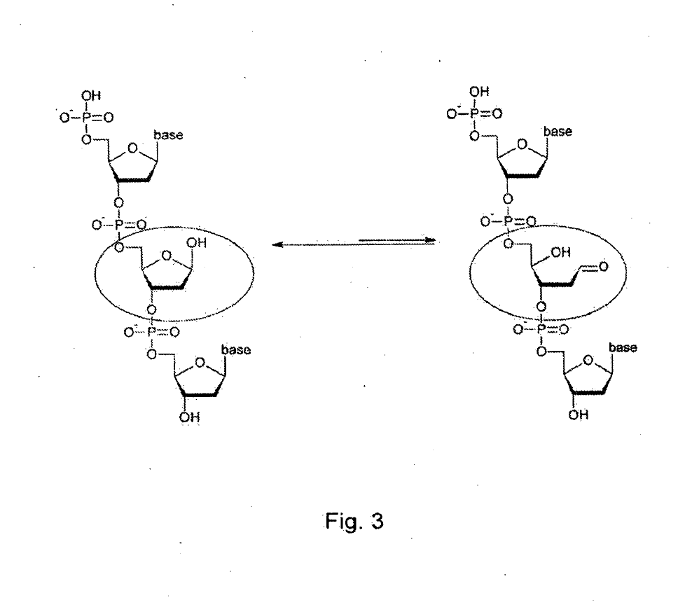

FIG. 4 illustrates the mechanism of temozolomide, (TMZ, 3,4-dihydro-3-methyl-4-oxoimidazo[5,1-d]-as-tetrazine-8-carboxamide) leading to methylated DNA bases.

FIG. 5 illustrates the condensation reaction between open ring aldehyde form of AP site and methoxyamine.

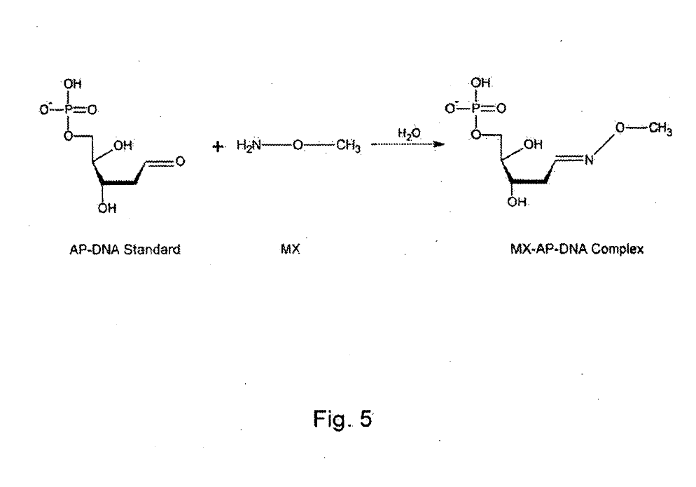

FIG. 6 illustrates the condensation reaction between an AP site and a FARP molecule.

FIG. 7 shows an energy level diagram of Fluorescence. The figure shows three different types of relaxations such as vibration relaxation, internal conversion, and fluorescence. Vibrational relaxation and internal conversion are shown in nonradioactive relaxation.

FIG. 8 shows a block diagram of an exemplary fluorometer for assaying of DNA utilizing methods disclosed herein.

FIG. 9 is a graph illustrating the excitation and emission spectra of FARP 25 mM in 10 mM phosphate buffer at pH 9.0.

FIG. 10 is a graph illustrating a calibration curve of FARP in 10 mM phosphate buffer (pH 9.0). The curve plots individual FARP standard solutions from 0.100 to 1000 nM. The fluorescence intensity of each individual FARP standard solution was plotted against its concentration after subtracting the fluorescence of the blank of phosphate buffer (pH 9.0).

FIG. 11 is a graph illustrating the fluorescence intensities of the condensation product under different pH and time at 37° C. [AP-DNA]=150 nM, [F121]=50 μm reaction buffer: 10 mM phosphate buffer at pH 5.6, 6.5, and 7.0; detection buffer 10 mM phosphate buffer at pH 9.0.

FIG. 12 is a graph illustrating the fluorescence intensities of the condensation product under different pH and time at room temperature. [AP-DNA]=150 nM. [F121]=50 μm reaction buffer: 10 mM phosphate buffer at pH 5.6, 6.5, and 7.0; detection buffer 10 mM phosphate buffer at pH 9.0.

FIG. 13 is a graph illustrating the effect of [FARP] on fluorescence intensity of the condensation product. The assay procedures were the same as those described in the Experimental by varying FARP concentration.

FIG. 14 is graph illustrating a reversibility study of the condensation reaction [★]; AP-DNA without subsequent incubation, []; Blank AP-DNA.

FIG. 15 is a graph illustrating ratios of [FARP]/[MX] in a Competition Study where MX and FARP were competing for a limited amount of AP-DNA and only FARP-AP-DNA was fluorescent.

FIG. 16 is a graph illustrating the calibration curve of AP-DNA standards. [∘] denoted the fluorescence intensity of the blank AP-DNA solution.

FIG. 17 is a pair of graphs illustrating AP-site profiles obtained by (A) ARP assay and (B) FARP assay using SW480 cell line.

FIG. 18 is a pair of graphs illustrating AP-site profiles obtained by (A) ARP assay and (B) FARP assay using human nonadherent cells.

FIG. 19 is a graph illustrating reduced fluorescence intensity in the presence of MX and MX-like compounds.

### DETAILED DESCRIPTION

The present invention relates to a method of assaying DNA apurinic/apyrimidinic abasic (AP) sites in a biological sample, such as a biological sample that is obtained from a subject. The method can be used to detect and/or quantitate AP-sites in DNA isolated from the biological sample to determine the effectiveness of various DNA damaging agents, such as anti-neoplastic agents and anti-mitotic agents.

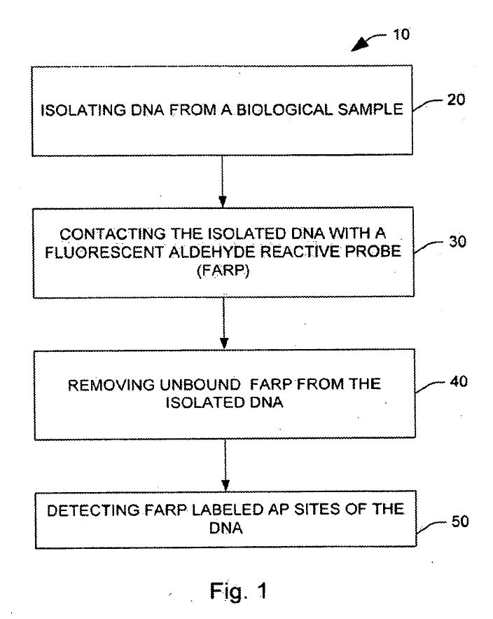

FIG. 1 is schematic flow diagram illustrating a method 10 in accordance with an aspect of the invention. In the method 10, at 20 a sample of DNA is isolated from a biological sample, such as a biological sample obtained from a subject under examination and/or a subject treated with a DNA damaging agent, such as an anti-neoplastic agent and/or anti-mitotic agent. The biological sample obtained from the subject can include blood, tissue, as well individual cells. In one example, the sample of DNA can be isolated from peripheral blood mononuclear cells obtained from a subject.

The DNA sample can be isolated from the biological sample using conventional DNA isolation and purification methods. Conventional approaches to DNA isolation and purification are based on multi-step procedures involving phenol/chloroform (see for example Sambrook. J. et al, Molecular Cloning: A Laboratory Manual, 2^{nd} Edition, Cold Spring Harbor Laboratory Press, 1989). An example of a commonly used method for isolating DNA from a DNA source, e.g., blood, saliva, tissue samples, etc., involves lysing the DNA source with a combination of a proteolytic enzyme and a detergent followed by extracting the mixture with an organic solvent, e.g., phenol and chloroform, so that the DNA enters the aqueous phase and the hydrolyzed products enter the organic phase. The DNA in the aqueous phase is then precipitated by the addition of alcohol.

In another approach, DNA can be isolated by lysing the DNA source with a chaotropic substance, for example guanidinium salt, urea and sodium iodide, in the presence of a DNA binding solid phase. The released DNA is bound to the solid phase in a one step reaction, where the beads are washed to remove any residual contaminants. Although these methods have proven to be less time consuming and toxic, they have resulted in a moderate level of DNA shearing and some level of contamination. In a further approach, a sample of DNA can be isolated from a starting source by mixing the starting source with a cationic detergent, which forms a hydrophobic complex between the DNA and detergent. The hydrophobic complex is separated from the solubilized contaminants and the DNA recovered by addition of a salt.

Following isolation of the DNA from the biological sample, at 30, the isolated DNA sample is contacted with a fluorescent aldehyde reactive probe (FARP) that binds to apurinic/apyrimidinic (AP) sites of the DNA. The FARP includes a fluorescent portion and a binding portion. The fluorescent portion can include any fluorescent molecule that upon exposure to or excitement by light of specific wavelength is capable of fluorescing. In one example, the fluorescent portion is visible light excitable. The binding portion can include any molecule that is capable of reacting with an aldehyde group of an AP site of the DNA to form a covalent bond and bind the FARP to the AP site. The binding portion can include for example an amine, aminoxy, hydrazone, hydrazine, hydroxylamine, and/or derivative thereof.

In an aspect of the invention, the FARP can be a fluorescent hydrazine, fluorescent hydroxylamine, or a derivative thereof that is capable of reacting with aldehydes, such as those exposed on AP-sites. The fluorescent hydrazine, fluorescent hydroxylamine, and/or derivative thereof can be visible light-excitable.

One example of a fluorescent aldehyde reactive reagent is fluorescein-5-thiosemicarbazide. It will be appreciated that the present invention is not limited to the use of fluorescein-5-thiosemicarbazide and that other fluorescent hydrazine, fluorescent hydroxylamine, or derivatives thereof may be utilized in the methods of the present invention. Examples of fluorescent hydrazines, fluorescent hydroxylamines, and derivatives thereof excited with visible light include Alexa Fluor 448, Alexa Fluor 555, Alexa Fluor 568, Alexa Fluor 594, Alexa Fluor 633, Alexa Fluor 647 hydrazides and hydroxylamines, BODIPY FL hydrazide, and Texas Red Hydrazide.

The FARP can be provided in a buffer, such as a phosphate buffer to form a FARP reagent. In one example, about 5 μM FARP can be included in about 100 μL of aphosphate buffer (10 mM, pH 7.0). An excess amount of the FARP reagent can be contacted with a sample of DNA taken from a subject in order to assure that each available AP-site comes in contact with FARP.

At 40, following contact of the DNA sample with the FARP, excess FARP reagent can then be removed from the sample to avoid background fluoresence. The excess FARP can be removed by washing the sample without detaching the DNA bound FARP. For example, an about 70% ethanol solution can be added to the sample, removed, and then discarded. The sample may be washed one or more times in order to assure removal of unbound FARP reagent.

At 50, following removal of the excess FARP, the FARP labeled AP sites of the DNA sample can be detected and quantitated. The FARP labeled AP sites can be quantitatively detected fluorometrically or through other types of electromagnetic spectroscopy, which analyze fluorescence from the sample. Devices that measure fluorescence are commonly referred to as fluorometers, fluorimeters, or fluorescence spectrophotometers. An example of a fluorimeter for use in the present invention is the Fluoromax-2 fluorimeter (ISA-SPEX, Edison, N.J.). The Fluoromax-2 or similar device can detect very low levels of fluorescence in samples.

The measured fluorescence can be compared with the fluorescence of standard control specimens of known AP-DNA concentrations to quantitate or determine the number of AP sites in the DNA sample. Blank AP-DNA background readings from the control DNA can also be used to quantitatively determine the number of AP sites of the DNA sample. In an aspect of the present invention, the concentration of AP sites in the DNA sample can be quantitatively determined by plotting the fluorescence intensity versus the concentration of AP sites of the DNA sample. The concentration AP sites of the DNA sample can then be correlated with the concentration of plotted AP sites of the control specimen to determine the amount of AP-DNA in the isolated DNA from the biological sample.

To produce control DNA of known AP-DNA concentrations against which the sample of DNA can be compared, double stranded calf thymus can be obtained and specific numbers of AP sites can be selectively produced, as known in the art. Typically a heat/acid depurination buffer treatment can be used to produce useful control samples. As described in Example 1 herein, multiple working solution AP-DNA controls of varying concentrations can be produced and utilized in the methods provided. In one embodiment of the present invention, controls and samples can be assayed by treating the sample of DNA and control DNA specimens in parallel so that the sample and control DNA specimen(s) are each subjected to the same or similar environmental and process conditions so as to remove any such variables from the respective samples when interpreting the results of their comparisons. The ordering of the steps included in the present invention may be altered. For example, the sample of DNA and control DNA specimens may be labeled separately with FARP reagent, and then fluorescently measured and compared.

The present invention also relates to a kit for assaying AP site of a DNA sample. The kit can include a control DNA specimen having a known concentration of AP-sites and a FARP reagent. In an aspect of the invention, the kit can also include instructions to explain how one may fluorometrically compare a given sample of DNA and control DNA. The instructions can further include directions on contacting the sample DNA and a set of control DNA specimens each having a known number of AP sites with FARP reagent. The kit may also include further instructions on performing fluorometric analysis to correlate the amount of AP-sites in a sample of DNA relative to the control DNA specimens.

The present invention further relates to a screening assay that can be used to screen for compounds that inhibit repair of AP sites of injured or damaged DNA, such as by base excision repair (BER). Base excision repair (BER) is initiated during replication of DNA and allows for correction of damaged bases/mispaired bases prior to completion of replication. In single-nucleotide BER, the deoxyribose phosphate (dRP) in the abasic site is removed by the lyase activity of DNA pol β. Compounds such as methoxyamine can react with the aldehyde of an AP site, making it refractory to the β-elimination step of the dRP lyase mechanism, thus blocking single-nucleotide BER. AP endonuclease inhibitors may act by binding to AP sites and preventing APE-mediated cleavage of phosphodiester bonds, or by acting directly on AP endonuclease.

In an aspect of the present invention, the screening assay can be used for identifying compounds that are capable of binding (e.g., covalent binding) with an aldehyde group on an AP site of the DNA. Examples therapeutic agents can include an AP endonuclease inhibitor. Compounds useful as BER inhibitors include AP endonuclease inhibitors such as methoxyamine (MX), N-ethylmaleimide, O⁶-benzylguanine, and their derivative compounds. It is not intended that the present invention be limited by the nature of the agents screened in the screening assay of the present invention. A variety of compounds, including peptides, organic compounds, nonorganic compounds, as well as, formulations of more than one compound, are contemplated.

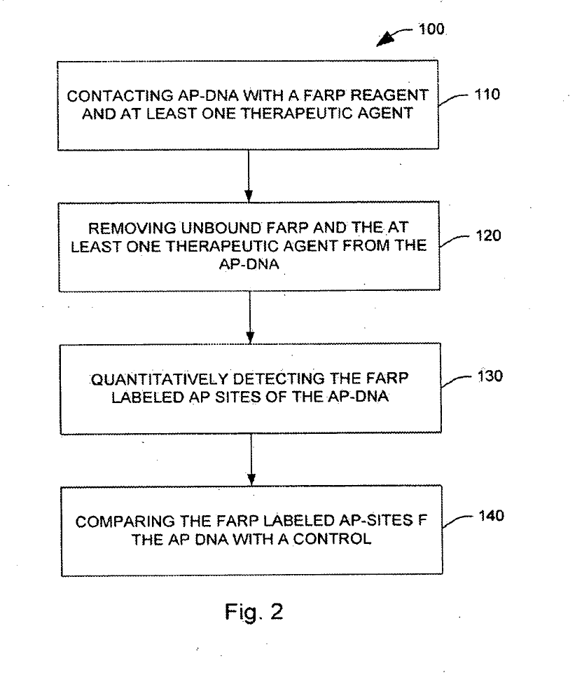

FIG. 2 illustrates a schematic flow diagram of a method 100 of identifying compounds that inhibit repair of AP sites of injured or damaged DNA. In a method 100, at 110, an FARP reagent and one or more therapeutic agents to be screened for the ability to bind to the aldehyde group on an AP site of the DNA are combined with a DNA specimen with AP sites (i.e., AP-DNA). The DNA specimen can be control DNA specimen, such a control DNA specimen of the method 10.

At 120, unbound FARP reagent and unbound therapeutic agent are removed from the sample of AP-DNA, such as by washing the sample. For example, an about 70% ethanol solution can be added to the sample, removed, and then discarded. The sample may be washed one or more times in order to assure removal of unbound FARP reagent and the therapeutic agent.

At 130, following washing the number of FARP labeled AP-sites is detecting in the sample of AP-DNA. The FARP labeled AP sites can be quantitatively detected fluorometrically or through other types of electromagnetic spectroscopy, which analyze fluorescence from the sample.

At 140, following measurement of the fluorescence of the sample, the measured fluorescence can be compared with the fluorescence of a control DNA samples, which has combined with the FARP reagent but not the therapeutic agent to quantitate or determine the binding efficiency or level of therapeutic agent to AP sites in the DNA sample. A lower level of FARP labeled AP-sites detected in the presence of an agent compared to a level detected with FARP alone may be indicative of a useful therapeutic agent for inhibiting BER.

Various other assays may be practiced using the analysis methods hereof. The above are only exemplary. This invention is further illustrated by the following examples which should not be construed as limiting.

### Example

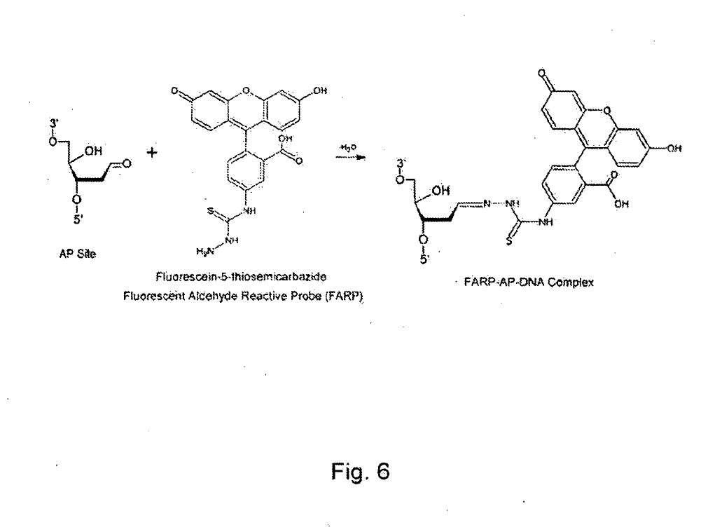

In this work, a direct fluorometric assay has been developed for quantitative determination of DNA-AP sites. The assay exploits the condensation reaction between an AP site and a fluorescein-5-thiosemicarbazide molecule (a fluorescent aldehyde reactive probe, FARP) (FIG. 6). After precipitation and re-suspension FARP labeled DNA, the fluorescence intensity is measured and the concentration of AP sites in the DNA sample is determined using a calibration curve. Compared to the ARP, this assay is rapid, simple and precise. Furthermore, because of its direct measurement, the assay can be used not only as a quantitative method, but also as a screening method for AP site blocking agents, which permits us to screen therapeutic agents for the blockage of BER.

### Principle of Fluorescence

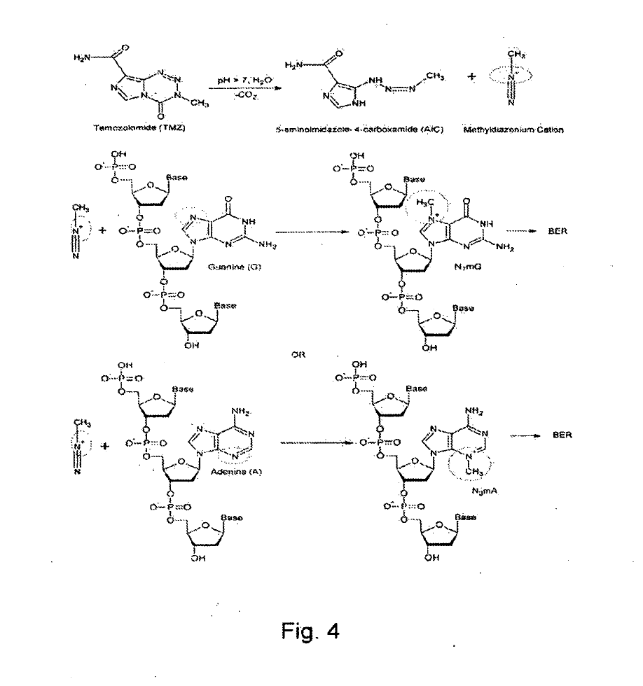

Chemical compounds absorb energy that excites electrons in the molecule, such as increased vibrational energy or transitions between discrete electronic energy states under suitable environment. The absorbed energy must be equal to the difference between the ground energy electronic state and the excited higher electronic state for the transition to occur. The energy difference of the excitation wavelength is constant and distinctive for the molecular structure. In FIG. 4, E₀ is the ground state, and E₁ and E₂ are electronic excited states. Each of electronic state has four excited vibration states. The excited molecule would return to the ground state (Eₒ) through emission of energy in form of heat and/or emission of quanta such as photons. The emission wavelength also corresponds to the difference between two different electronic energy states. Thus, it could be also the characteristic of the molecular structure (Skoog et al. (1996) Fundamentals of Analytical Chem. 7^{th} Ed. 601-607).

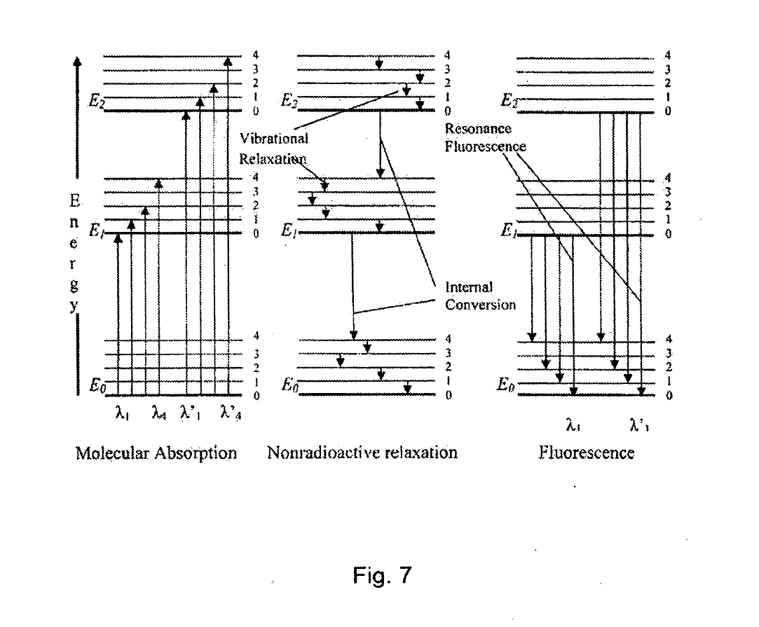

Fluorescence occurs when a molecule emits photons as it returns from the excited states to its ground state after the absorption of photons from 200-900 nm (Skoog et al. (1996)). Fluorescence could be shown in three steps. First, energy absorption through irradiation that the molecule will be in different excited states according to the amount of energy from irradiation. Higher irradiation can excite the molecule into higher energy level. Then the relaxation process will happen to release excess energy. There are three different types of relaxations such as vibrational relaxation, internal conversion, and fluorescence. Vibrational relaxation and internal conversion are shown in nonradioactive relaxation (FIG. 7).

Vibrational relaxation takes place during collisions between excited molecules and solvents. The excess vibrational energy is transferred into solvent molecules in a series of steps during collision, which cause a slight temperature increase of the solution. The average life time of vibrational state is only for 10⁻¹⁵ s. Nonradioactive relaxation occurs between the lowest vibrational level of an excited level (E₂ in FIG. 7) and the upper vibrational level of another excited state (E₁ in FIG. 7). The internal conversion is much less efficient than vibrational relaxation. The average time of the electronic excited states is between 10⁻⁶ and 10⁻⁹ (Skoog et al. (1996)).

Fluorescence is the relaxation process from the lowest vibrational level of an excited electronic state to the any vibrational levels of the ground electronic states. Due to the vibrational relaxation and internal conversion, a Stokes' shift is observed in fluorescence. In the other word, the energy is lost through heat or vibration, so that emitted energy is less than the exciting energy. Therefore, the emission wavelength is longer than the excitation wavelength due to lower energy.

The excitation spectrum and emission spectrum usually have approximate mirror images because the energy differences between vibrational states are about the same for both ground state and excited state.

Because of the properties of emission and excitation wavelength of fluorescent compounds, fluorometric technique can be used for qualification and quantification. The key advantage of fluorescence over radioactivity and absorption spectroscopy is said to be the ability to separate compounds on the basis of either excitation or emission spectra, as opposed to a single spectra.

The effect of concentration on fluorescence intensity can be determined through the following equation, F=2.3K′εbcP₀ (Guilbalt (1990) Modern Monographs in Anal. Chem. 3^{rd} Ed. 25-46). F is the power of fluorescent radiation, while P₀ is the power of the beam incident on the solution, and P is its power after the beam traverses a length b. And symbol c is the concentration of fluorescing particles. The equation can be written as F=Kc at constant P₀. Based on this equation, a plot of the fluorescence intensity of a sample in solution versus the concentration of the emitting species is linear at the lower concentration. At very high concentration, F can reach the maximum and even decreases with increasing concentration (Skoog et al. (1996), Guilbalt (1990)).

At high concentrations of molecules or with very short path lengths, fluorescence intensity corresponding to the concentration decreases due to quenching. While the concentration of molecules in a solution increases, the excited molecules would interact with each other and lose energy through processes other than fluorescent emission. The reduction of the probability of fluorescent emission is known as quenching. Quenching can occur because of the presence of impurities in the sample or in the cell, increasing temperature, or reducing viscosity of the solution media.

### Instrumentation

The fluorescence spectrophotometer consists of a light source, slits, excitation monochromator, sampling compartment, reference detector, emission monochromator and detector (Skoog et al. (1996)).

A source of fluorescence spectrophotometer must generate a sufficient beam power at wavelength region of interest. Also a source should provide a stable output during the measurement. FluoroMax-2 has a xenon light source that provide continuous source. The bandwidth of slit can range from 0-30 nm depending on the signal strength. Usually, with low concentration samples, slit width goes wide to collect more light. On the other hand, narrower slit width is used to avoiding too high signal responding. Once the broad range of light is provided from the light source, a monochromator transmits a selected wavelength through a mechanical selection of a narrow band.

In common Czerny-Turner design, the light source is focused on to the entrance slit. The intensity of source can be set with slit and the angle of the optical system. The slit should be effectively located to focus a collimator (a curved mirror) that collimates the light through refraction of light from the slit. Then the collimated light is either refracted with prism or diffracted from grating, which is changing the direction of light. Subsequently, the light is collected by the second mirror. This second mirror can refocus and collect light on to the slit. Both entrance and emission slits can be adjusted.

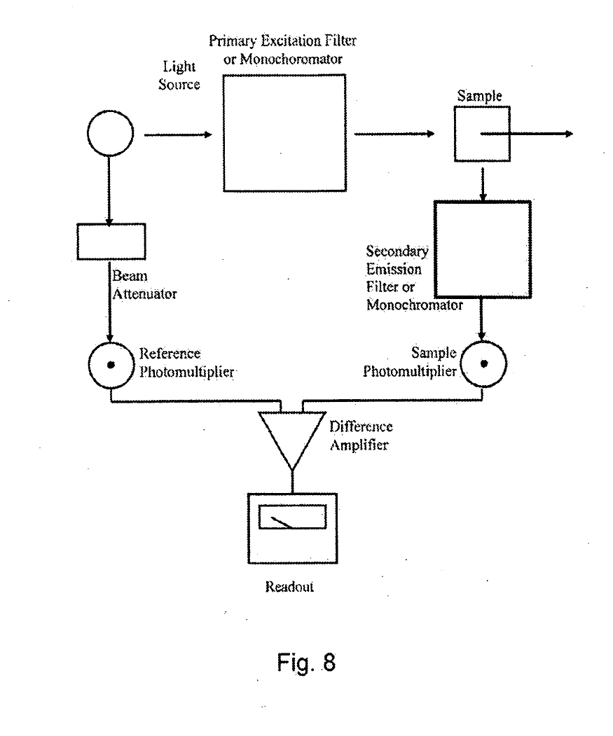

After the light goes through the excitation monochromator at selected excited wavelength, it passes through sample to excite fluorophores in the sample solution. Then the fluorescent illumination goes to the emission monochromator that has the same mechanical design as the excitation monochromator. Finally, the fluorescence intensity is determined by an emission detector. The simple block diagram follows in FIG. 8 (Skoog et al. (1996)).

### Chemicals and Materials

Calf thymus DNA (Cat. No. D-4522), monosodium phosphate, disodium phosphate, N,N-dimethylformamide (DMF, 99.9+%), methoxyamine hydrochloride (MX.HCl), ethoxyamine hydrochloride, benzyloxyamine hydrochloride, 4-fluorophenylhydrazine hydrochloride, phenoxyamine hydrochloride, ammonium acetate, chloroform, bovine serum albumin, and sodium dodecyl sulfate were purchased from Sigma-Aldrich (Milwaukee, Wis., USA). Fluorescein-5-thiosemicarbazide (FARP) (Cat. No. F 121) was from Molecular Probe (Eugene, Oreg., USA). Tris(hydroxymethyl) aminomethane (TRIS) and ethylenediaminetetraacetic acid disodium salt dehydrate (EDTA) were obtained from Bio-Rad Laboratories (Hercules, Calif., USA). Temozolomide (TMZ) was provided by the Developmental Therapeutics Program of the National Cancer Institute (Rockville, Md., USA).

ARP (N′-aminoxyethylcarbonylhydrazino D-biotin) solution (Cat. No. DKO2-000) and ARP-labeled DNA standard solution set (Cat. No. DKO2-101) were from Dojindo Molecular Technologies (Gaithersburg, Md., USA). Proteinase K (Cat. No. 25530-015) was from Invitrogen (Carlsbad, Calif., USA). OPTITRAN-BA-S85 0.45-tm reinforced nitrocellulose membrane (Cat. No. 10439262) and minifold II vacuum filter device were from Schleicher and Schuell (Keene. NH, USA). Peroxidase-conjugated streptavidin (Cat. No. HK330-5K) was from BioGenex (San Ramon, Calif., USA). Amersham enhanced chemiluminescence (ECL™) substrate (Cat. No. RPN 2106) was purchased from GE Healthcare (Piscataway, N.J., USA). EPPENDORF Safe-Lock Tubes (1.5 mL) (Brinkmann Instruments, Westbury, N.Y., USA) were used for sample storage and assay reactions.

### Solutions

Deionized water was obtained from a NANOpure system (Barnstead, Dubuque, Iowa, USA) and sterilized by autoclave before use.

The stock solutions of methoxyamine (MX, 100 mM), MXL101 (10.0 mM), MXL102 (10.0 mM), MXL103 (10.0 mM) and MXL104 (10.0 mM) were prepared by dissolving proper amount of each individual compound in the form of hydrochloride in a known volume of deionized water. The pH values of these stock solutions were adjusted to 7.0 using 1 M NaOH prior to the complete addition of water. The working solutions of methoxyamine (0.0100, 0.100, 1.00, 3.00 and 12.5 mM), MXLIO1 (0.0100, 0.100 and 1.00 mM), MXL1O2 (0.0100, 0.100 and 1.00 mM), MXL103 (0.0100, 0.100 and 1.00 mM), and MXL1 04 (0.0100, 0.100 and 1.00 mM) were prepared by serial dilutions of the stock solutions with phosphate buffer (10.0 mM, pH 7.0).

The FARP stock solution (50.0 mM) was prepared by dissolving proper amount of FARP in 70% DMF aqueous solution (v/v). The FARP stock solution (500 μM) was prepared by 1:100 dilution from the 50.0 mM FARP stock solution with 70% DMF aqueous solution (v/v) and divided into 100-μL aliquots. The FARP working solution (10.0 μM) was prepared by 1:50 dilution of the 500 μM FARP stock solution with phosphate buffer (10.0 mM, pH 7.0)

Proteinase K (10 mg/ml), 10% SDS, ammonium acetate (2.0 and 7.5 M), phosphate buffer (10 mM, pH 7.0 & pH 9.0), TE Buffers (10 mM TRIS-HCl/1 mM EDTA, pH 7.0 & 9.0), and sodium citrate buffer (0.3 M of sodium citrate, 3 M NaCl, pH 5.0), PBS (lx, pH 7.4: 2.7 mM KCl, 1.8 mM KH₂PO₄, 137 mM NaCl, 10.1 mM Na₂HPO₄), SSC (20×, pH 7.0: 0.300 M sodium citrate and 3.00 M NaCl), SSC (5×, pH 7.0: 0.075 M sodium citrate, 0.75 M NaCl) were prepared by dissolving proper amount of chemicals in deionized water and adjusting pH to the desired values.

Saturated phenol solution (Cat. No. BP 17501-400) was from Fisher Scientific (Fair Lawn, N.J., USA), which was supplied at pH 6.6, together with a bottle of 1 M TRIS buffer. The saturated phenol solution was adjusted to pH 7.9 using the TRIS buffer and used for DNA purification in this work.

All stock solutions were kept in the freezer (−20° C.) before use.

### Apparatus and Instrumentation

Model 4810 EPPENDORF autoclavable pipettes were used for transferring samples, solutions and solvents.

A FLUOROMAX-2 spectrofluorometer (Jobin Yvon Inc., Edison, N.J., USA) with DataMax (version 2.20) software was used for this work. Standard and sample solutions were measured using a 160-iL fluorometer cell (Cat. No. 16.160F-Q-10/Z8.5) produced by Starna Cells (Atascadero, Calif., USA). The instrument should be turned on 30 minutes prior to the measurements. Calibration of the instrument had been performed on regular basis (once per month or as needed) using both air and water. For the quantitation of FARP labeled DNA-AP sites, the excitation and emission wavelengths were set at 492 and 513 nm. The slit widths were 3.5 nm for both emission and excitation, and the integration time was 0.5 s. The spectrofluorometer should be turned on 30 mm prior to the measurements and calibration should be done at least once per month.

An HP8453 UV-visible spectrophotometer (Hewlett-Packard, Wilmington, Del., USA) and an HP PC with ChemStation software were used for the measurement of DNA concentrations. A Digital Heatblock I (VWR Scientific Products, West Chester, Pa., USA), a Microfuge 22R Centrifuge (Beckman Coulter, Palo Alto, Calif., USA), and a DNA 120 SpeedVac (Thermo Servant, Holbrook, N.Y., USA) were also used for DNA and AP-DNA preparation and isolation.

### Selection of Emission and Excitation Wavelength

To select the emission and excitation wavelength of FARP, a simple experiment was performed. The excitation spectrum was obtained by setting the emission wavelength at 516 nm while scanning the excitation wavelength from 350 to 500 nm; whereas the emission spectrum was resulted by setting the excitation wavelength at 492 nm while scanning the emission wavelength from 505 to 650 nm.

### Sample Preparation and Handling

Spectrofluorometry is a sensitive analytical technique. During sample measurements, one should pay attention to the details of sample preparation and handling, as well as operation of the instrument.

In this work, preparation of homogeneous DNA solution was crucial for correct sample measurements because the DNA samples were quite viscous. From one sample to another, the fluorometer cell (cuvette) needed to be washed thoroughly (water, 2 times; methanol, 3 times) or sample carryover could occur.

Contamination could lead to misreading. Residue of organic products such as lab gloves could give fluorescence response during a measurement. In addition, adsorption of dust or fiber on the cell, as well as air bubbles, could scatter excitation and emission lights. While the above errors could be easily determined in a scan mode, it could not be easily recognized when fixed wavelengths were used.

Since micro pipettes were used for every sample and solvent measurements, they were calibrated periodically with analytical balance by weighing water delivered.

### Preparation of AP-DNA Standards

### AP-Site Free DNA Stock Solution.

The AP-site free DNA stock solution was prepared by dissolving 3.52 mg calf thymus DNA from the original bottle in 5.00 mL of 1.00 mM methoxyamine working solution. The resultant AP-site free DNA stock solution (0.704 mg/mL) was pippetted into ten 1.5-mL EPPENDORF tubes at 500 j.tL each and kept in the freezer (−70° C.) before use.

### Blank AP-DNA Stock Solution.

The blank AP-DNA stock solution was prepared by following steps: (a) added 250 μL of the AP-site free DNA stock solution into a 175-μL ammonium acetate solution (7.5 M) and mixed; (b) added 1000 μL of ice-cold ethanol (100%) to the solution from (a), mixed by inverting the solution slowly, then kept at −20° C. for 30 min; (c) centrifuged the precipitated DNA at 4° C. and 12,000 g for 10 min; (d) discarded the supernatant and kept the DNA pellet; (e) added 1000 μL of ice-cold ethanol (70%) to the DNA pellet, vortexed, and centrifuged at 4° C. and 12,000 g for 10 mm; then discarded the supernatant; (f) repeated step (e) twice; (g) left the wet DNA precipitate in the tube to dry at room temperature for 30 min in a venting hood; and (h) hydrated the dried DNA precipitate in 500 μL phosphate buffer (10 mM, pH 7.0) and resuspended the DNA by vortexing. This blank AP-DNA stock solution was kept in the freezer (−70° C.) before use.

The absorbance of the blank AP-DNA stock solution was measured by UV-visible spectrophotometer after a 1:100 dilution with 10 mM phosphate buffer at pH 7.0. Using the equation: [DNA] (mg/mL)=A_{260 nm}×50× dilution factor, and the ratio of A_{260 nm}/A₂₈₀=1.8 or higher for highly purified DNA solution (Note: protein contamination in the DNA sample may cause a positive error) (Aposhian et al. (1962) J. Biol. Chem. 237:519-525), the concentration of the blank AP-DNA solution was calculated to be 332 μg DNA/mL. Assuming 660 μg DNA per pmol of DNA (at size of 106 bp), the molar concentration of blank AP-DNA stock solution was 503 pM DNA (at size of 106 bp).

### AP-DNA Standard Stock Solution.

To prepare the AP-DNA standard stock solution, the dried blank AP-DNA precipitate was first obtained through the steps (a) to (g) in the section of “Blank AP-DNA Stock Solution”. The AP-DNA standard stock solution was prepared using the heat/acid depurination procedure as follows (Eshleman et al. (1995)): (a) resuspended the dried DNA precipitate in 50 μL of the sodium citrate buffer (0.3 M of sodium citrate, 3 M of NaCl, pH 5.0); (b) depurinated the DNA sample at 70° C. for 30 min using a VWR Digital Heatblock I and filled the tube holders with water to keep the temperature constant; (c) cooled the AP-DNA solution rapidly in an ice bath; (d) added 1000 μL of ice-cold ethanol (100%) to the solution from (c), and mixed by inverting the solution slowly before putting it at −20° C. for 30 mm; (e) centrifuged the precipitated AP-DNA at 4° C. and 12,000 g for 10 min; (f) discarded the supernatant and kept the AP-DNA pellet; (g) added 1000 μL of ice-cold ethanol (70%) to the AP-DNA pellet, vortexed, and centrifuged at 4° C. and 12,000 g for 10 min; then discarded the supernatant; (h) repeated the step (g) twice; (i) left the AP-DNA precipitate in the tube to dry at room temperature for 30 mm in a venting hood; and (j) hydrated the dried AP-DNA precipitate in 500 μL phosphate buffer (10 mM, pH 7.0) and resuspended it by vortexing. This standard stock solution was kept in the freezer (−70° C.) before use.

The absorbance of the AP-DNA standard stock solution was measured by UV-visible spectrophotometer after a 1:100 dilution with 10 mM phosphate buffer at pH 7.0, and the concentration of the AP-DNA standard stock solution was calculated to be 330 μg DNA/mL, which corresponding to a molar concentration of 500 μM DNA (at size of 106 bp). The AP-DNA standard stock solution was analyzed by the ARP assay which gave a result of 911 AP sites per double-stranded DNA molecule (at the size of 106 bp). Therefore, the AP-DNA standard stock solution had a concentration of 456 nM.

### AP-DNA Standard Working Solutions.

The AP-DNA standard working solutions (2.50, 5.00, 10.0, 20.0, 40.0, 80.0, 160, and 320 nM) were prepared as follows. 640 nM AP-DNA standard was first prepared by diluting 351 μL of 456 nM AP-DNA standard stock solution to 500 μL using phosphate buffer (10 mM, pH 7.0), and the remaining standards were prepared by 1:1 dilution of the standard working solution at higher concentration with phosphate buffer (10 mM, pH 7.0).

### AP-Site Assays

### ARP Assay.

An ARP method modified for the slot-blot vacuum filter device was used in this work. The procedure of the ARP assay was as follows: (a) added 15 μL ARP solution to 10 μg DNA extracted from cells in a tube, adjusted the total volume to 150 μL with PBS (lx, pH 7.4) and incubated at 37° C. for 15 min; (b) prepared blank AP-DNA control by following the same procedure as (a); (c) added 400 μL ice-cold ethanol (100%), mixed gently by inverting the tube, and placed the tube in −20° C. for 30 min; (d) centrifuged the precipitated DNA at 4° C. and 12,000 g for 10 min; (e) discarded the supernatant and kept the DNA pellet; (f) washed the DNA pellet with 400 μL of ice-cold ethanol (70%) by vortexing, then centrifuged at 4° C. and 12,000 g for 10 min, and discarded the supernatant; (g) left the wet DNA precipitate in the tube to dry at room temperature for 30 min in a venting hood; and (h) hydrated the dried DNA precipitate in 100 μL TE buffer (pH 7.0) at room temperature for 1 hr or at 4° C. overnight and resuspended the DNA by vortexing; (i) pipetted 3 μL the resuspended DNA solution and diluted to 100 μL with TE buffer (pH 7.0), which gave a concentration of 0.3 μg/100 μL; (j) placed the Optitran®-BA-S85 0.45-μm reinforced nitrocellulose membrane in SSC (20×) for 1 hr, and denatured the DNA samples by heating them at 100° C. for 5 mm, then chilling on ice; (k) added 100 μL ammonium acetate (2.0 M) to each sample and vortexed; (l) assembled the slot-blot device by placing 2 pieces of Whatman paper which were pre-soaked in SSC (20×) below the nitrocellulose membrane; (m) put each DNA sample into a well and applied vacuum until the entire sample solution had passed through the membrane, and wash each well with vacuum using 200 μl ammonium acetate (2.0 M); (n) baked the membrane in an 80° C. oven for 1 hr (after this step, the membrane could be stored at 4° C. overnight); (o) submerged the membrane in 100 μL of 0.25% BSA/PBS containing horseradish peroxidase-conjugated streptavidin (1:100) at room temperature, and shook for 40 min; (p) washed the membrane in the washing buffer for 1 hr using a shaker; (q) combined ECL reagent 1 and 2 in 1:1 ratio, and placed the membrane in ECL solution for 1 min; (r) drained the excess ECL solution, wrapped the membrane in a plastic wrap, and taped it in an X-ray cassette; (s) placed an X-ray film behind the membrane and closed the cassette lid in the dark room with light off; (t) exposed the X-ray film to light for 1 s to 10 min (depending on the darkness of bands on the film, which was generally done through the prior knowledge by trial and error); (u) removed the film and developed it in an X-ray film developer; and (v) scanned the film using a scanner into the computer, and determined the band intensity using the NIH Image J software.

Quantification of AP sites by the ARP assay could be done by constructing a calibration curve using ARP-labeled DNA standards.

### FARP Assay.

100 μL of 5 μM FARP in phosphate buffer (10 mM, pH 7.0) was added to 200 μL phosphate buffer (10 mM, pH 7.0) containing 10 μg of DNA extracted from cells, individual AP-DNA standard (10, 20, 40, 80, 160, 320, and 640 nM AP), and blank AP-DNA control, respectively. After vortexing, the reaction was allowed to proceed at room temperature for 15 min. At the end of the reaction 200 μL ammonium acetate (7.5M) and 1000 μL ice-cold ethanol (100%) were added. The solution was mixed gently by inverting the tube before keeping it at −20° C. for 30 min. Each tube was centrifuged at 4° C. and 12,000 g for 10 min. The supernatant was poured out carefully so that no loss of FARP-AP-DNA took place. 1200 μL of ice-cold ethanol (70%) was added to each tube to remove the free FARP by centrifugation at 4° C. and 12,000 g for 10 min. This washing step with 70% ice-cold ethanol was repeated two more times. After discarding the supernatant, the FARP-AP-DNA precipitate in the tube was dried at room temperature for 30 min. in a venting hood. The FARP-AP-DNA precipitate was hydrated and resuspended in 200 μL of phosphate buffer (10 mM, pH 9.0) at room temperature for 1 hr. The fluorescence intensity of the resultant FARP-AP-DNA solution was measured by the FluoroMax-2 spectrofluorometer. A calibration curve could be constructed by plotting the fluorescence intensity versus the concentration of AP sites.

### Cells and Drug Experiments

Adherent SW480 colon cancer cells (from the American Type Culture Collection (Manassas, Va., USA), and human nonadherent mononuclear cells from heparinized peripheral blood of a normal donor, which were purified by the Ficoll-Hypaque density gradient centrifugation technique, were used in the in vitro experiments of TMZ and MX.

The cells (5×10⁶ cells) were plated in 100-mm culture dishes and treated by TMZ alone at various concentrations (0, 187, 375, 750, and 1500 μM), and together with MX (12.5 mM) for 2 hrs at 37° C. At the end of treatment, the cells were washed by either rinsing the SW480 cells on dishes (SW480) or centrifugation of the human cells in tubes with the media. After plating the cells in dishes with fresh media, 3 mM MX was added to the dishes that were treated with TMZ and MX together. The cells were harvested by centrifugation at 24 after the treatment of 3 mM MX.

Extraction of DNA from Cells

The nuclear DNA extraction procedure included the following steps: (a) collected the cells and spun them down; (b) kept the cell pellet in −20° C. until use; (c) resuspended the cell pellet in 2.0 mL TE buffer (pH 7.0), added 0.24 mL of 10% SDS, and mixed well; (e) added 12.5 μL of 10 mg/mL proteinase K, mixed well and incubated overnight at 37° C.; (f) added 2.0 mL of saturated phenol solution (pH 7.9), mixed gently by inverting the tube, and placed the tube in a shaker for 30 min; (g) centrifuged at 10,000 g for 10 min, and removed the upper aqueous phase containing DNA to a fresh tube; (h) repeat the step (g); (i) if a white interface was visible after the second extraction, repeated the step (g) again; (j) added 2 mL chloroform, mixed gently by inverting the tube, and placed the tube in a shaker for 30 min; (k) centrifuged at 10,000 g for 10 min, and removed the upper aqueous phase containing DNA to a fresh tube; (l) Added 1.0 mL ammonium acetate (7.5 M), and mixed well; (m) added 10.0 mL of ice-cold ethanol (100%) to the solution, mixed gently by inverting the tube, then kept at −20° C. for 30 mill; (n) centrifuged the precipitated DNA at 4° C. and 12,000 g for 10 min; (o) discarded the supernatant and kept the DNA pellet; (p) added 2.0 mL of ice-cold ethanol (70%) to the DNA pellet, vortexed, and centrifuged at 4° C. and 12,000 g for 10 min; then discarded the supernatant; (q) repeated the step (p) twice; (r) left the wet DNA precipitate in the tube to dry at room temperature for 30 min or longer in a venting hood; and (s) resuspended the dried DNA precipitate in 50-100 μL phosphate buffer (10 mM, pH 7.0) (the volume was dependent upon the size of DNA precipitate); and (t) determined the DNA concentration using UV-vis spectrophotometer after 1:10 dilution with the phosphate buffer (10 mM, pH 7.0).

### Results and Discussion

### The Excitation and Emission Wavelength of FARP

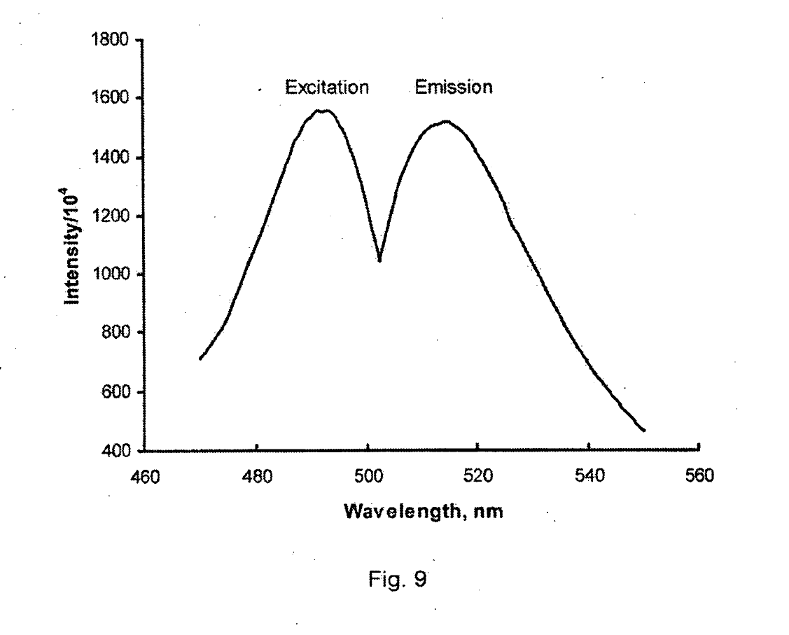

FIG. 9 combined the excitation and emission spectra for 25 mM FARP in 10 mM phosphate buffer at pH 9.0. In order to bring these two spectra in one graph, a multiplication factor of 700 was applied to the excitation readings. From FIG. 9, the maximum excitation and emission wavelengths were indicated to be 492 and 513 nm, respectively. In comparison with the reference values provided by the manufacturer (492 nm for excitation and 516 nm for emission), the 3-nm blue shift for the maximum emission wavelength was probably caused by the matrix of the solution.

### Linear Calibration Range of FARP

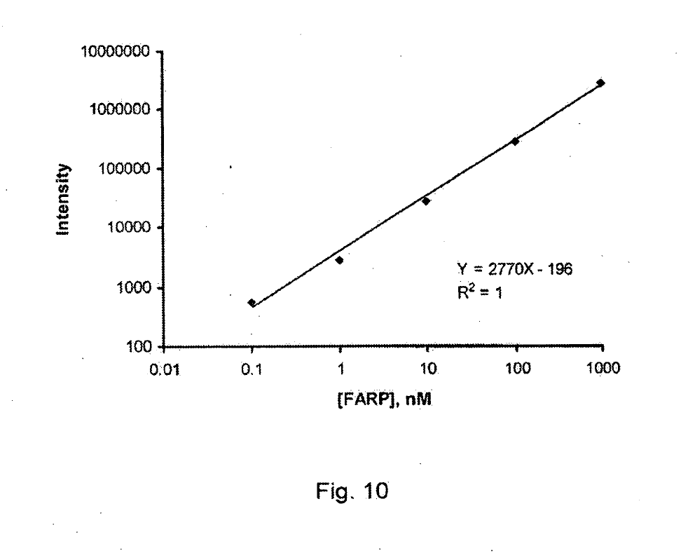

The fluorescence intensity versus the FARP concentration was investigated in this study. The results showed that a wide linear dynamic range could be obtained for FARP from 0.100 to 1000 nM with a correlation coefficient of 1.00 in 10 mM phosphate buffer at pH 9.0 (Table I and FIG. 10). In the measurement, the phosphate buffer (pH 9.0) was used as the blank. For constructing a calibration curve, the fluorescence intensity of each individual FARP standard solution was plotted against its concentration after subtracting the fluorescence intensity of the blank (<300). The pH value of 9.0 was chosen for fluorescence measurement because it had been reported that FARP produces the highest fluorescence emission under pH 9.0.

TABLE I
Calibration of FARP in 10 mM phosphate buffer (pH 9.0)

| [FARP], nM | Fluorescence Intensity |
|---|---|
| 0.100 | 561 |
| 1.00 | 2,778 |
| 10.0 | 27,368 |
| 1.00 × 10² | 276,230 |
| 100 × 10³ | 2,707,173 |

### Effect of pH, Time and Temperature on the Reaction of FARP and AP-DNA

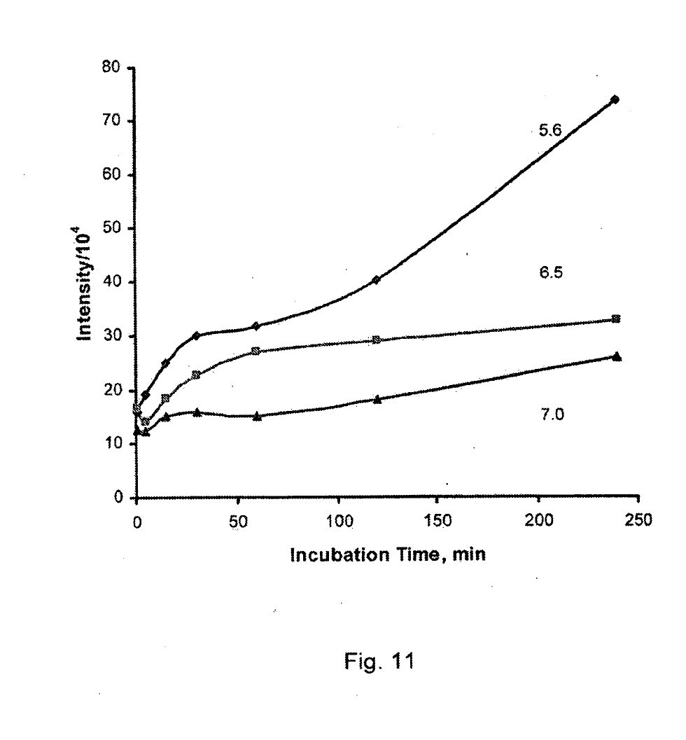

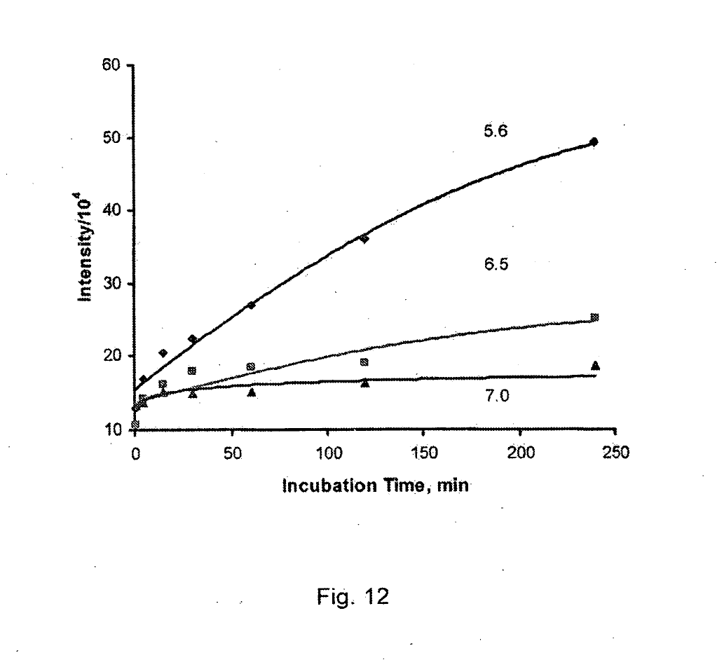

The condensation reaction between AP-DNA and FARP (FIG. 8) was affected by the pH of the solution and the reaction time, as well as the reaction temperature. In this work, three pH conditions at 5.6, 6.5 and 7.0 of 10 mM phosphate buffer were tested for the condensation reaction over a period of 240 mm under two temperature settings (room temperature and 37° C.). The fluorescence responses of the reaction product were measured in 10 mM phosphate buffer at pH 9.0 and the data were shown in Table II and FIG. 11, and Table III and FIG. 12.

As shown in FIGS. 11 and 12, a lower pH value of 5.6 and a higher temperature of 37° C. produced higher fluorescence intensities than those at pHs 6.5 and 7.0, as well as those at the room temperature. These figures revealed that lower pH value and higher temperature favored the condensation product. Furthermore, the fluorescence intensities of the condensation product at pH 5.6 increased drastically from the time point of 60 mm, and this increase was much greater at 37° C. than that of the room temperature, which implied that there were new AP sites generated during the condensation reaction due to further acid/heat depurination of the AP-DNA under the chosen experimental conditions.

FIGS. 11 and 12 also showed that a plateau of fluorescence intensity could be reached at pH 7.0 at the time course of 15-60 mm for the room temperature and 37° C. Therefore, a reaction time of 15 mm and a pH 7.0 buffer were chosen for the FARP assay for its condensation reaction.

TABLE II
The fluorescence intensity data of condensation product
under different pH and time at 37° C.

| | Fluorescence Intensity | | |
|---|---|---|---|
| Time (min.) | pH 5.6 | pH 6.5 | pH 7.0 |
| 1 | 159,591 | 165,434 | 126,004 |
| 5 | 190,389 | 140,938 | 122,264 |
| 15 | 249,566 | 183,772 | 151,363 |
| 30 | 299,529 | 225,276 | 157,363 |
| 60 | 317,724 | 270,212 | 151,235 |
| 120 | 402,476 | 290,265 | 182,267 |
| 240 | 737,944 | 328,034 | 258,721 |

TABLE III
The fluorescence intensity data of condensation product
under different pH and time at the room temperature.

| | Fluorescence Intensity | | |
|---|---|---|---|
| Time (min) | pH 5.6 | pH 6.5 | pH 7.0 |
| 1 | 130,199 | 109,916 | 134,290 |
| 5 | 168,278 | 142,110 | 137,770 |
| 15 | 204,475 | 160,989 | 151,081 |
| 30 | 224,416 | 178,965 | 149,149 |
| 60 | 268,882 | 185,553 | 150,489 |
| 120 | 359,852 | 189,760 | 163,256 |
| 240 | 492,657 | 251,437 | 186,291 |

### Effect of FARP Concentration on Fluorescence Intensity

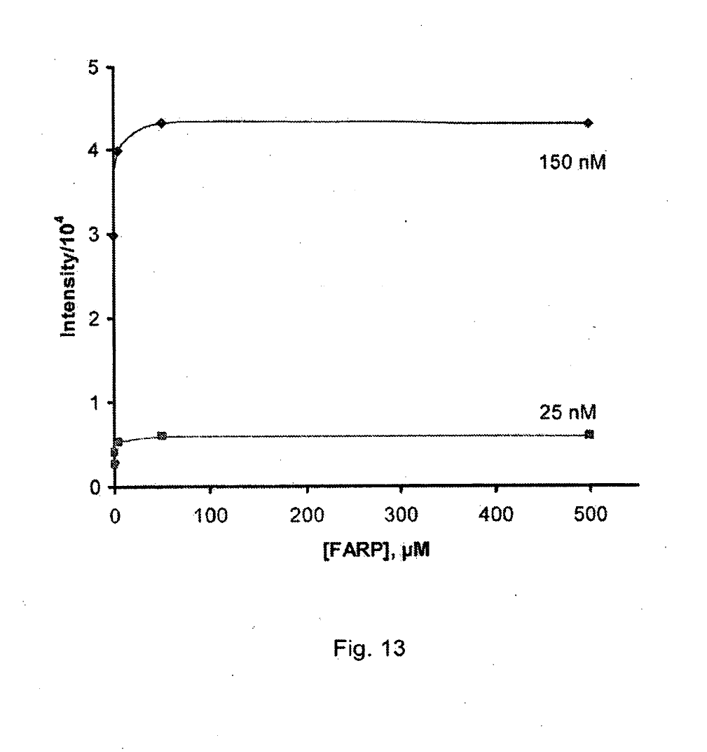

The effect of FARP concentration (0.0500-500 μM) on the rate of condensation reaction between FARP and AP-DNA was investigated. As shown in Table IV and FIG. 13, when the AP-DNA concentration remained constant at 25 and 150 nM, the fluorescence intensity of the condensation product increased as the FARP concentration increased, and reached plateaus at concentration of 5.00 mM for 25.0 nM AP-DNA and 50.0 μM for 150 nM AP-DNA. Therefore, a FARP concentration of 50.0 μM was used in the procedures of the FARP assay.

TABLE IV
[FARP] versus the Fluorescence Intensity of the Condensation Product

Fluorescence Intensity

| [F121] (μM) | [AP-DNA] (25 nM) | [AP-DNA] (150 nM) |
|---|---|---|
| 0.0500 | 2,763 | 2,889 |
| 0.500 | 3,987 | 29,781 |
| 5.00 | 5,216 | 39,786 |
| 50.0 | 5,873 | 43,219 |
| 500 | 5,873 | 43,250 |

Reversibility of the Condensation Reaction between FARP and AP-DNA

It was known that the aldehyde group of an AP site could easily condense with aminooxy or hydrazone nucleophiles. However, the reversibility of the reaction was not clear, especially in the presence of another competing nucleophile. To clarify the above, two experiments were designed and carried out as follows: (a) FARP (50 μM in 100 μL of 10 mM phosphate buffer at pH 7.0) reacted with AP-DNA (125 nM in 200 μL of 10 mM phosphate buffer at pH 7.0) for 1 hr, after washing the condensation product (FARP-AP-DNA) incubated with MX (50 μM in 100 μL of 10 mM phosphate buffer at pH 7.0) for various time periods; and (b) MX (50 μM in 100 μL of 10 mM phosphate buffer at pH 7.0) reacted with AP-DNA (125 nM, 200 μL) for 1 hr, after washing the condensation product (MX-AP-DNA) incubated with FARP (50 μM in 100 μL of 10 mM phosphate buffer at pH 7.0) for various time periods.

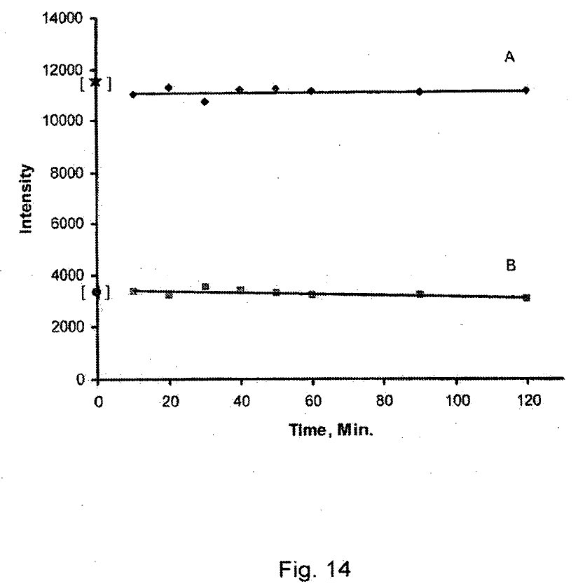

The results were given in Table V and FIG. 14. Since there were neither the replacement of FARP by MX (FIG. 12, Curve A), nor the replacement of MX by FARP (FIG. 14, Curve B) observed following the subsequent incubations, it was concluded that the condensation reaction between aminooxy or hydrazone nucleophiles and aldehyde group of AP-DNA is irreversible under the assay conditions. This unique feature of the assay can be used to screen MX-like compounds in chemotherapeutic development.

TABLE V
Reversibility Study of the Condensation Reaction

| | | |
|---|---|---|
| | FARP Reaction | MX Replacement |
| | Followed | Followed by |
| Replacement | MX Reaction | FARP Replacement |

| | |
|---|---|
| Time (min) | Fluorescence Intensity |

| | | |
|---|---|---|
| Blank AP-DNA (60) | 3,512 | 3,512 |
| AP-DNA without | 11 487 | |
| subsequent | | |
| incubation | | |
| 10 | 11,003 | 3,366 |
| 20 | 11,289 | 3,225 |
| 30 | 10,759 | 3,557 |
| 40 | 11,193 | 3,410 |
| 50 | 11,226 | 3,321 |
| 60 | 11,139 | 3,192 |
| 90 | 11,112 | 3,202 |
| 120 | 11,145 | 3,081 |

Competitive Reactions Between FARP and MX with AP-DNA

Since aminooxy and hydrazone nucleophiles can react with aldehyde group of AP site and form condensation product, it is desirable to know when MX and FARP compete with AP-DNA, which compound has greater reactivity toward AP-DNA. The finding of this experiment will lay the foundation of a screening assay for MX-like compounds.

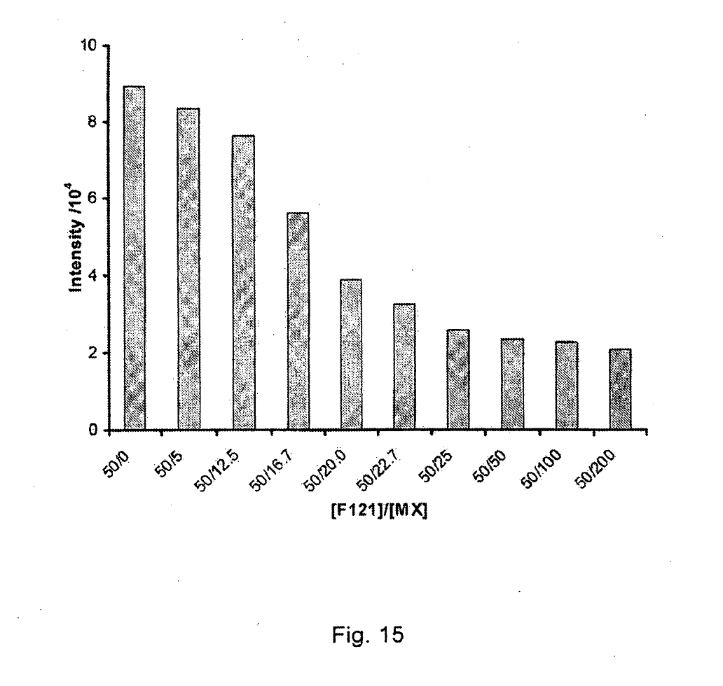

In the study, the amount of AP-DNA used were 125 nM in 200 μL of 10 mM phosphate buffer at pH 7.0 and the ratios of [FARP]/[MX] were as follows: 50 μM FARP/0 μM, 50.0 μM/5 μM, 50.0 μM/12.5 μM, 50.0 μM/16.7 μM, 50.0 μM/25.0 μM 50.0 μM/22.7 μM, 50.0 μM/25 μM, 50.0 μM/50.0 μM, 50.0 μM/100.0 μM, 50.0 μM/200.0 μM, 50.0 μM/500.0 μM in 100 μL of 10 mM phosphate buffer at pH 7.0. Because MX and FARP were competing for a limited amount of AP-DNA, and only FARP-AP-DNA was fluorescent, the reduced fluorescence intensity reflected the amount of MX binding to the AP sites. As shown in Table VI and FIG. 15, MX showed a greater affinity toward APDNA than that of FARP. This is probably due to the molecular size of MX, which allows it to diffuse to faster to the AP-DNA than FARP molecule in aqueous solution.

TABLE VI
Data of Competitive Reactions between FARP and MX with AP-DNA

| [FARP]/[MX] | Fluorescence Intensity/10⁴ |
|---|---|
| 50.0 μM/0.0 μM | 8.9754 |
| 50.0 μM/5.0 μM | 8.9453 |
| 50.0 μM/12.5 μM | 8.3567 |
| 50.0 μM/16.7 μM | 7.6453 |
| 50.0 μM/20.0 μM | 5.6423 |
| 50.0 μM/22.7 μM | 3.8976 |
| 50.0 μM/25.0 μM | 3.2452 |
| 50.0 μM/50.0 μM | 2.587 |
| 50.0 μM/100.0 μM | 2.348 |
| 50.0 μM/200.0 μM | 2.2876 |
| 50.0 μM/500.0 μM | 2.0896 |

### Linear Calibration Range and Limit of Detection of FARP AP-Site Assay

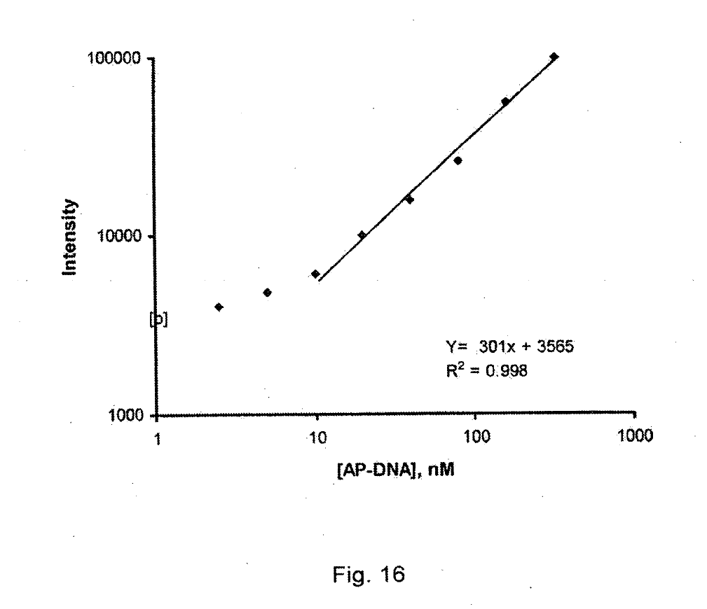

AP-DNA standard solutions were prepared in 10 mM phosphate buffer (pH 7.0) according to the procedure described in the Experimental section. The blank AP-DNA used was 200 mL of 351 pM, which was prepared by dilution of the blank AP-DNA stock solution in 10 mM phosphate buffer at pH 7.0. The linear calibration range the AP-site assay was investigated using AP-DNA standards ranged 2.50-320 nM. As shown in Table VII and FIG. 16, the calibration range was 10.00-320 nM AP-DNA with a correlation coefficient of 0.998. The blank AP-DNA denoted as [o] in FIG. 16 gave a mean fluorescence intensity of 3324 (n=3). The limit of detection of the FARP AP-site assay was defined as the mean fluorescence intensity plus 3× standard deviation of the blank AP-DNA, which was calculated to be 1.04 nM.

TABLE VII
[AP-DNAJ versus Fluorescence Intensity of the Assay

| [AP-DNA] (nM) | Fluorescence Intensity |
|---|---|
| Blank (n = 3) | 3,324 (n3) |
| 2.5 | 3,992 |
| 5 | 4,824 |
| 10 | 6,134 |
| 20 | 9,986 |
| 40 | 15,643 |
| 80 | 25,549 |
| 160 | 54,871 |
| 320 | 98,786 |

### Reproducibility

The intra-day and inter-day assay precision were investigated and calculated. For the intra-day assay precision, three parallel AP-DNA standards from each of the three concentration levels (20, 80, and 160 nM) were assayed within the same day. As shown in Table VIII, the overall intra-day assay precision presented by R.S.D. was ≦4%. For the inter-day assay precision, three parallel AP-DNA standards from each of the three concentration levels (15.6, 62.5 and 125 nM) were measured in separate days (Table VIII), which had the overall inter-day assay precision in R.S.D. 9%.

TABLE VIII
The intra- and inter-assay of AP-DNA (n = 3)

| Within-Day | | | |
|---|---|---|---|
| (n = 3) | [AP-DNA], nM | Mean ± S.D. (n = 3) | R.S.D. (%) |
| | 160 | 44,785 ± 1,221 | 3 |
| | 80.0 | 25,357 ± 1,120 | 4 |
| | 20.0 | 8,355 ± 339 | 4 |
| Between-Day | | | |
| (n = 3) | [AP-DNA], nM | Mean ± S.D. (n 3) | R.S.D. (%) |
| | 125 | 43,008 ± 1,732 | 7 |
| | 62.5 | 23,953 ± 1,644 | 7 |
| | 15.6 | 6,278 ± 582 | 9 |

### Applications of FARP Assay in Development of Chemotherapeutic Agents Quantitative Measurement of AP Sites in Drug-Induced DNA Damage

TMZ is a methylating agent that can damage cancer cells by linking its methyl group to DNA bases. However, the DNA damages can be repaired by DNA repair systems in cells, resulting in drug resistance. A novel anticancer agent, MX, is currently under clinical investigation for overcoming cancer resistance to TMZ. The proposed molecular mechanism of MX is to react with AP sites produced by DNA glycosylase and block the further repair of AP endonuclease in the DNA base excision repair system.

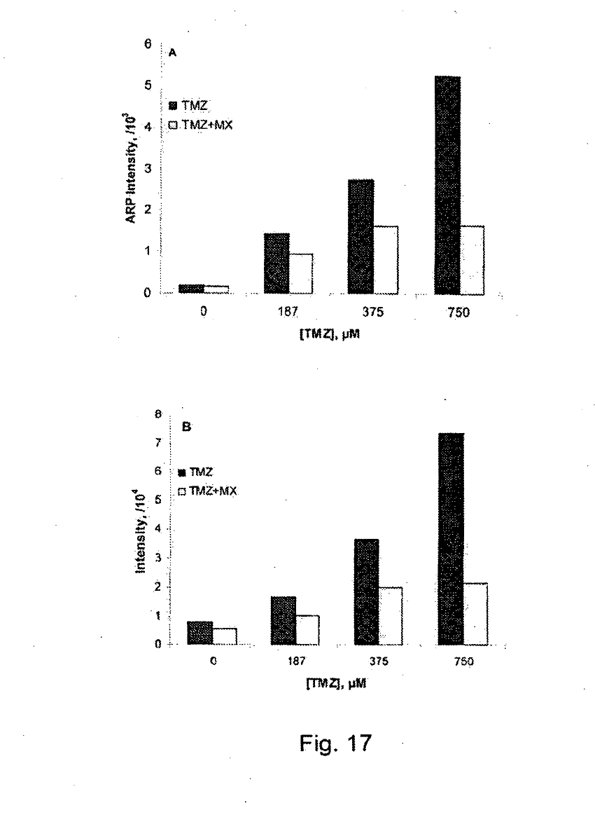

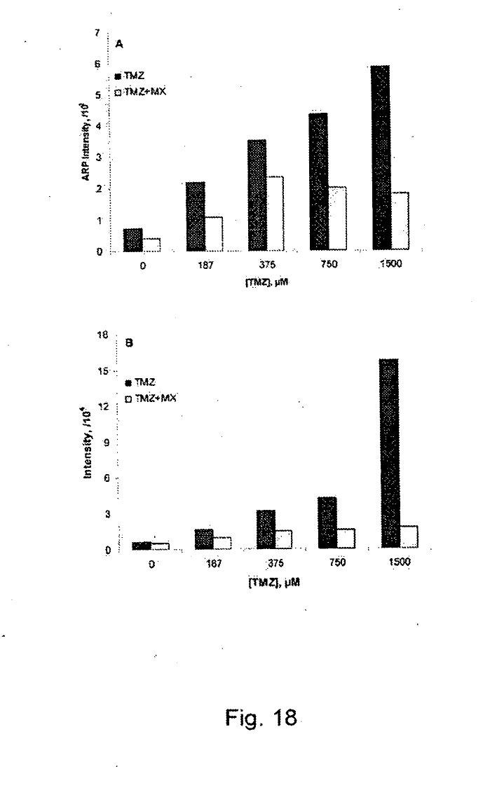

To correlate the efficacy of TMZ with and without MX, the quantitative measurement of AP sites were conducted using SW480 colon cell line and human nonadherent mononuclear cells (see Experimental section for the details). The DNA samples were analyzed by both ARP and FARP assays, and results were shown in Table IX and FIG. 17 (SW480 cell line), and Table X and FIG. 18 (human cell). Although both assays showed comparable AP-site profiles in two cell experiments, several distinctive differences were worth noting. Firstly, the FARP assay sensitivity was found to be greater than that of ARP assay due to larger signal change in FARP assay; secondly, the signal differences between TMZ and TMZ+MX were greater for FARP assay than that of ARP assay; thirdly, the FARP assay had greater specificity and reproducibility toward TMZ and TMZ+MX treated DNA samples that those of ARP assay; and finally, the FARP assay is more efficient in terms of simplicity, time and cost in comparison with the ARP assay.

TABLE IX
Data obtained from ARP and FARP assays using SW480 cell line.

| | |
|---|---|
| ARP Assay | FARP Assay |
| Band Intensity/10³ | Fluorescence Intensity/10⁴ |

| TMZ | | TMZ + 12.5 | | TMZ + 12.5 |
|---|---|---|---|---|
| (μM) | TMZ | mM MX | TMZ | mM MX |
| 0.00 | 0.198 | 0.180 | 0.645 | 0.486 |
| 187 | 1.420 | 0.950 | 2.428 | 1.150 |
| 375 | 2.723 | 1.617 | 3.691 | 1.492 |
| 750 | 5.233 | 1.644 | 5.310 | 1.999 |

TABLE X
Data obtained from ARP and FARP assays
using human nonadherent cell.

| | ARP Assay | | | |
|---|---|---|---|---|
| | Band Intensity/10³ | | FARP Assay | |
| | | TMZ + 12.5 | Fluorescence | TMZ + 12.5 |
| TMZ(μM) | TMZ | mMMX | Intensity/10⁴ TMZ | mM MX |
| 0.00 | 0.721 | 0.420 | 0.649 | 0.532 |
| 187 | 2.201 | 1.095 | 1.622 | 0.957 |
| 375 | 3.524 | 2.336 | 3.138 | 1.510 |
| 750 | 4.352 | 2.002 | 4.201 | 1.577 |
| 1500 | 5.844 | 1.797 | 15.77 | 1.746 |

### Screening of MX-Like Compounds

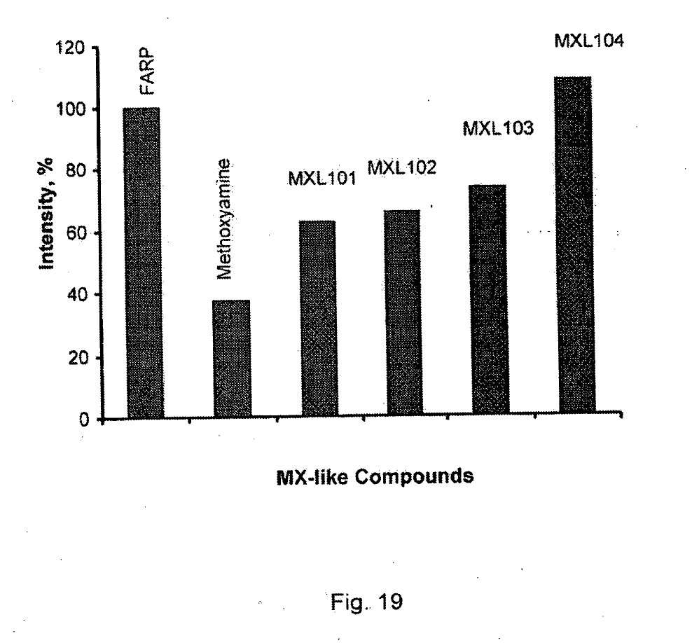

One important application of the FARP AP-site assay developed is to screen MX-like compounds for their reactivity to AP site based on known chemical structures. In this work, four compounds with aminooxy or hydrazone group (i.e., MXL1O1, MXLIO2, MXLI 03, and MXL 104) together with MX were tested. The detail experiment included mixing equal concentration and volume of each individual test compound with FARP (50 μM in 100 μL of 10 mM phosphate buffer at pH 7.0) before reacting with 200 L of 100 nM AP-DNA. The remaining experimental procedures were the same as described in the Experimental section. The data of fluorescence intensity reduced by MX and MX-like compounds were given in Table XI and the fluorescence intensity of the test compounds were compared in FIG. 19.

As shown in Table XI and FIG. 19, MX had higher reactivity than FARP toward AP-DNA because the recovery of fluorescence intensity (37.3%) was small than 50% (where equal reactivity was found). MXL 101, MXL 102, and MXL 103 showed lower reactivity than FARP but in competition with FARP with the recovery of fluorescence intensity ranged 62.4-73.4%. Furthermore, MXLI 04 was non-reactive toward AP-DNA with the recovery of fluorescence intensity of 107.8%. The over 100% recoveries of fluorescence intensity were probably due to the experimental deviation. This work proved that the FARP AP site assay could be used to screen AP-site reactive compounds for targeting DNA base excision repair pathway in chemotherapeutic development.

TABLE XI
Data of Fluorescence Intensity Reduced by MX-like Compounds

| | | | Normalized |
|---|---|---|---|
| | Fluorescence Intensity | | Fluorescence |
| Compounds | Individual Measurement | Mean | Intensity % |
| FARP | 17080.0, 16892.0 | 16986.0 | 100.0 |
| Methoxyamine | 6543.0, 6137.0 | 6340.0 | 37.3 |
| MXL101 | 10560.0, 10627.0 | 10593.5 | 62.4 |
| MXL102 | 10723.0, 11543.0 | 11133.0 | 65.5 |
| MXL103 | 12793.0, 12133.0 | 12463.0 | 73.4 |
| MXL104 | 18037.0, 18570.0 | 18303.5 | 107.8 |

### CONCLUSION

A direct fluorometric assay for quantitative measurement of AP site and screening of AP-site reactive compounds has been developed using fluorescein-5-thiosemicarbazide as the fluorescent aldehyde reactive probe (FARP). This assay has a linear dynamic range of 10-320 nM AP-DNA with a calibration equation of Y=301X+3565 and a correlation coefficient of 0.998. The limit of detection defined as the blank plus 3 times of its standard deviation was 1.04 nM AP-DNA. The intra-day and inter-day assay precisions were calculated as ≦4 and ≦9% (R.S.D.). This assay has been applied to the analysis of nuclear DNA damage after treatment of cells with TMZ, and TMZ plus MX. In comparison to ARP assay, the newly developed FARP assay has greater assay performance in terms of specificity, sensitivity and reproducibility. It is a simple, rapid and cost effective assay. Furthermore, the newly developed FARP assay has been proven useful for screening AP-site reactive compounds owing to its direct assay format. It provides a high-throughput platform for screening of MX-like compounds in chemotherapeutic development.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

Having described the invention, the following is claimed:

1. A method of detecting abasic (AP) sites in DNA from a biological sample, the method comprising:
isolating a sample of DNA from the biological sample;
contacting the DNA with a fluorescent aldehyde reactive probe (FARP); and
detecting FARP labeled AP sites in the DNA sample.

2. The method of claim 1, the DNA being extracted from a subject's cells before the contacting step.

3. The method of claim 1, the FARP comprising fluorescein-5-thiosemicarbazide.

4. The method of claim 1, further comprising the step of correlating the number of AP sites in the sample of DNA to the number of AP sites in a control DNA specimen.

5. The method of claim 4, the control DNA specimen comprising an AP-DNA standard having a known concentrations of AP sites.

6. The method of claim 1, the AP sites of the sample DNA and the control DNA specimen being determined substantially simultaneously.

7. The method of claim 1, further comprising removing unbound FARP from the sample of DNA after contacting the DNA with the FARP.

8. A method of quantitating AP sites in DNA isolated from a biological sample, the method comprising:
contacting the isolated sample of DNA with a FARP reagent;
removing unbound FARP from the sample of DNA; and
quantitatively assessing the number of AP sites in the sample of DNA.

9. The method of claim 8, the number of AP sites of the DNA sample being quantitatively assessed by fluorometric analysis.

10. The method of claim 9, wherein a fluorescence intensity of the sample of DNA is correlated to the concentration of AP sites in the sample of DNA by comparing the fluorescence intensity in the sample of DNA to the fluorescence intensity a control DNA specimen contacted with FARP.

11. The method of claim 8, the FARP reagent comprising fluorescein-5-thiosemicarbazide.

12. The method of claim 9, the control DNA specimen comprising an individual AP-DNA standard having a known concentration of AP sites.

13. A kit for assaying a sample of DNA comprising:
a control DNA specimen having a known concentration of AP sites; and
a FARP reagent.

14. The kit of claim 13, the FARP reagent comprising fluorescein-5-thiosemicarbazide.

15. A method of screening therapeutic agents for inhibiting base excision repair (BER), the method comprising:
contacting a sample of AP-DNA with an FARP reagent and at least one therapeutic agent;
removing unbound FARP reagent and the at least one therapeutic agent from the sample of AP-DNA; and
detecting FARP labeled AP sites in the sample of AP-DNA.

16. The method of claim 15, further comprising correlating a level of FARP labeled AP sites in the sample of AP-DNA to the level of FARP labeled sites in a control sample of AP-DNA that is contacted with FARP but is not contacted with the therapeutic agent.

17. The method of claim 16, wherein a reduced level of FARP labeled AP sites in the sample of AP-DNA compared to the level of FARP labeled AP sites in the control sample is indicative of an effective therapeutic agent or an effective combination of therapeutic agents.

18. The method of claim 15, the therapeutic agent comprising a DNA repair inhibitor.

19. The method of claim 18, the DNA repair inhibitor comprising a base excision repair inhibitor.

20. The method of claim 19, the base excision repair inhibitor comprising an AP endonuclease inhibitor.

21. The method of claim 15, the therapeutic agent comprising a compound capable of forming a covalent linkage with an aldehyde group on AP-DNA.

22. The method of claim 15, the FARP reagent comprising fluorescein-5-thiosemicarbazide.
